(19) United States
(12) Patent Application Publication
(10) Pub. No.: US 2025/0340885 A1
(43) Pub. Date: Nov. 6, 2025

(54) COMPOSITIONS AND METHODS FOR MODULATING CONDITIONS ASSOCIATED WITH ALTERED ATP8B1 FUNCTION

(71) Applicant: CLEVELAND STATE UNIVERSITY, CLEVELAND, OH (US)

(72) Inventor: KAILASH GULSHAN, LEWIS CENTER, OH (US)

(21) Appl. No.: 19/199,589

(22) Filed: May 6, 2025

Related U.S. Application Data
(60) Provisional application No. 63/643,128, filed on May 6, 2024.

(51) Int. Cl. C12N 15/113; A61K 31/145; A61K 31/5025; A61P 1/16; A61P 29/00

(52) U.S. Cl. CPC C12N 15/1137; A61K 31/145; A61K 31/5025; A61P 1/16; A61P 29/00; C12N 2310/11; C12N 2310/14; C12N 2320/31; C12Y 207/01063

(57) ABSTRACT

Disclosed herein are compositions, systems, and methods for diagnosing, treating, and/or ameliorating the symptoms of conditions and diseases associated with abnormal ATP8B1 function. The disclosed compositions, systems, and methods are based on the discovery that ATP8B1 demonstrates PIP2 flippase activity and is implicated in a number of inflammatory conditions and diseases, including progressive familial intrahepatic cholestasis type 1 (PFIC1).

### CROSS-REFERENCE TO RELATED APPLICATION

This application claims priority to and the benefit of U.S. Provisional application No. 63/643,128, filed on May 6, 2024, the content of which is hereby incorporated by reference in its entirety.

### GOVERNMENT LICENSE RIGHTS

This invention was made with government support from the NHLBI grant no. R01-148158 and American Heart Association: AHA-SDG-15SDG25710128 and AHA-TPA-23TPA1063910. The government has certain rights in the invention.

### TECHNICAL FIELD

The present disclosure is in the field of compositions and methods of providing one or more therapeutic interventions. More particularly, the present disclosure provides compositions and methods for diagnosing and treating inflammatory conditions.

### BACKGROUND

ATP8B1 is a human gene that encodes a phospholipid flippase, a protein involved in transporting phospholipids across cell membranes. ATP8B1 helps move phospholipids from one side of a cell membrane to the other. Mutations in the ATP8B1 gene are associated with progressive familial intrahepatic cholestasis (PFIC1), a rare liver disease that primarily affects children and young adults and characterized by cholestasis, hepatomegaly, and other complications. PFIC1 is inherited in an autosomal recessive manner, meaning that both parents must carry a mutated copy of the ATP8B1 gene for their child to be affected.

PFIC1 patients often experience cholestasis (bile flow blockage), jaundice, diarrhea, and failure to thrive, among other symptoms. In addition to disrupted liver functions, PFICl patients show characteristic extrahepatic manifestations such as pruritus, hearing loss and pancreatitis. The underlying mechanisms regulating the pleiotropic functions of ATP8B1 are not fully clear. As a result, medical treatment for PFIC1 is limited, and liver transplantation may be an option in severe cases.

### SUMMARY

The following is a brief summary of subject matter that is described in greater detail herein. This summary is not intended to be limiting as to the general inventive concepts or the scope of the claims.

ATP8b1 is a member of P4-type ATPases (P4 ATPases). Human mutations in ATP8b1 causes PFIC-1, a disease characterized by a compromised detergent-resistant state of the hepatic canalicular membrane, leading to bile salt mediated excessive cholesterol extraction from hepatocytes and liver damage. ATP8b1 is also expressed in extrahepatic tissues such as cholangiocytes, pancreas and intestine, and PFIC1 patients show extra-hepatic symptoms including pancreatitis, secretory diarrhea, and sporadic hearing loss. The phenotypic spectrum of PFIC1 mutations in humans ranges from severe to mild. Severe form of PFIC1 is characterized by infantile-onset cholestasis that quickly progress to liver cirrhosis. Most of the hepatic dysfunction in PFIC1 has been attributed to the altered canalicular plasma membrane asymmetry, but the mechanistic details are lacking

Disclosed herein are various technologies pertaining to treatment for liver conditions and associated diseases and conditions. In certain embodiments, the general inventive concepts contemplate compositions and methods for treating or reducing the symptoms of progressive familial intrahepatic cholestasis 1. Human PFIC1 patients often present with extra-hepatic inflammatory clinical features, such as steatohepatitis, fat malabsorption, diarrhea, pancreatitis, and atherosclerosis. Thus, the general inventive concepts also contemplate methods and compositions for treating steatohepatitis, fat malabsorption, diarrhea, pancreatitis, and atherosclerosis, among other symptoms/conditions discussed herein.

In addition to disrupted liver functions, PFIC1 patients show characteristic extrahepatic manifestations such as pruritus, hearing loss and pancreatitis. The underlying mechanisms regulating the pleiotropic functions of ATP8B1 were not previously fully understood. The general inventive concepts are based, in part, on the discovery that ATP8B1 plays a role in maintaining phosphatidylinositol-4,5-bisphosphate (PIP2) on the inner leaflet of the plasma membrane (PM). Applicants have shown that homozygous ATP8b1 knockout (ATP8b1^{-/-}) cells exhibited enhanced PIP2 exposure at the cell-surface, accompanied by a marked reduction in PIP2 levels on the inner leaflet of the PM. ATP8b1^{-/-} cells also showed a marked defect in flipping exogenous PIP2 into the PM, while flipping of PE or bulk-endocytosis were unaffected. ATP8B1 exhibited direct binding to PIP2 via a conserved PIP2 binding region in the P-loop region. Unbiased RNAseq analysis of ATP8b1^{-/-} cells showed alternations in signal transduction, phospholipid translocation, and LPS response pathways. Strikingly, treatment of ATP8b1^{-/-} monocytes or macrophages with LPS alone led to Nlrp3-inflammasome-independent cleavage of pyroptotic executor Gasdermin D (GsdmD). The mechanism of GsdmD cleavage in ATP8b1^{-/-} cells involved LPS access to cytoplasmic compartment and induction of non-canonical inflammasome. ATP8b1^{-/-} macrophages showed defects in efferocytosis and phagocytosis. The atomic-force microscopy showed PM remodeling in ATP8b1^{-/-} macrophages, leading to increased membrane elasticity vs. WT cells. Taken together, our data identifies ATP8b1 as the first PIP2 flippase and negative regulator of GsdmD cleavage.

In certain embodiments, the general inventive concepts contemplate a method of treating, preventing, inhibiting, or ameliorating the symptoms of progressive familial intrahepatic cholestasis 1 (PFIC1) in an individual in need thereof, the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

In certain embodiments, the general inventive concepts contemplate a method of treating, preventing, inhibiting, or ameliorating hepatocyte damage in an individual in need thereof, the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

In certain embodiments, the general inventive concepts contemplate a method of reducing inflammation in an individual suffering from symptoms of progressive familial intrahepatic cholestasis 1 (PFIC1), the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

Other aspects and features of the general inventive concepts will become more readily apparent to those of ordinary skill in the art upon review of the following description of various exemplary embodiments in conjunction with the accompanying figures.

### BRIEF DESCRIPTION OF THE DRAWINGS

These and other features of the present disclosure will become better understood with regard to the following description and accompanying drawings in which:

FIG. 1A is a graph showing cell-surface PIP2 levels. The levels were determined in RAW264.7 cells (WT or ATP8b1^{-/-}) by flow cytometry using FITC-labeled PIP2 antibody, with ±ABCA1 expression (induced by treatment with 300 μM Br-CAMP for 12 h), or pretreatments with ±PIP2 Grip, or ±PI3P Grip for 30 minutes (RFI, relative fluorescence intensity; n=6, mean±SD; **** show p<0.0001, n.s=non-significant by ANOVA posttest).

FIG. 1B is a model depicting PIP2 exposure and FITC-PIP2 antibody binding at the cell-surface (outer leaflet of the plasma membrane) in WT, ATP8b1^{-/-}, and ABCA1 expressing cells.

FIG. 1C is an image of confocal microscopy.

FIG. 1D is a graph showing quantification of binding of FITC-labeled PIP2 antibody at the cell-surface of HepG2 cells (WT or ATP8b1^{-/-}).

FIG. 1E is an image of confocal microscopy of HepG2 cells (WT or ATP8b1^{-/-}) stably transfected with PIP2 reporter (2X-PH-PLC-eGFP), showing PIP2 localization (green fluorescence) at the inner leaflet of the PM or redistribution to PIP2 reporter from PM to cytoplasmic region.

FIG. 1F shows a cell scan profile plots showing fluorescent intensity of PIP2 reporter at the PM and cytoplasmic region of WT-HepG2 cells.

FIG. 1G shows a cell scan profile plots for ATP8b1^{-/-} HepG2 cells.

FIG. 1H is a graph showing quantification of the % fluorescence intensity of PIP2-GFP reporter at the plasma membrane of WT vs. ATP8b1^{-/-} HepG2 cells, values are mean±SD; **** show p<0.0001 by t-test.

FIG. 2A is a schematic diagram showing experimental design for PIP2 flip in WT vs. ATP8b1^{-/-} cells.

FIGS. 2B and 2C show live cell imaging showing flip of exogenously added Bodipy-TMR-PIP2 (1 μM) into the cell membrane of HepG2-WT cells (B) or HepG2-ATP8b1^{-/-} cells (C) over time.

FIG. 2D is a graph showing the fluorescence intensity of Bodipy-TMR-PIP2 over time for HePG2-WT vs. ATP8b1^{-/-} cells. Values are mean intensity±S.E.

FIGS. 2E and 2F are images showing (E) bodipy-TMR-PIP2 (1 μM) flip into the plasma membrane of RAW264.7-WT or (F) RAW264.7-ATP8b1^{-/-} cells over time.

FIG. 2G is a graph showing the fluorescence intensity of Bodipy-TMR-PIP2 over time for RAW264.7-WT vs. ATP8b1^{-/-} cells. Values are mean intensity±S.E.

FIGS. 2H and 2I show live cell imaging of RAW264.7-WT vs. ATP8b1^{-/-} cells showing flip of Rhodamine PE (1 μM) over time.

FIG. 2J is a graph showing quantification of fluorescence intensity profile of Rhodamine PE over time in WT vs. ATP8b1^{-/-} cells. Values are mean intensity±S.E. The experiments were independently replicated in triplicates. Scale bar is 5 μm.

FIG. 3A shows putative PIP2 binding domain of ATP8b1 protein is aligned with PIP2 binding domain of known PIP2-binding proteins.

FIG. 3B shows multiple sequence alignment of P2 region of P-loop of ATP8b1 (AA 812-847) showing the conserved PIP2 binding region across different species.

FIGS. 3C and 3D show Surface Plasmon Resonance (SPR) analysis of PIP2 binding with WT-ATP8b1 (C) or PBD mutant-ATP8b1 (D).

FIGS. 3E and 3F are plots showing microscale thermo-phoresis traces showing the relative fluorescence of Alexa-488 labeled WT-ATP8b1 (E) or Alexa-488 labeled PBD mutant-ATP8b1 (F).

FIG. 3G is a graph MST binding curve of Alexa-488 labeled WT-ATP8b1 or PBD mutant-ATP8b1 binding to DOPC:PIP2 (95:5) liposomes. The X-axis shows increasing concentration of DOPC:PIP2 (95:5) liposomes. Data plotted are mean±S.E.

FIG. 3H shows representative fluorescence spectra of 0.5 μM of Alexa488-labeled WT-ATP8b1 (donor)±1 mole % Rhodamine-PE (acceptor) doped in 200 μM of DOPC:PIP2 liposomes.

FIG. 3I is a graph showing FRET sensitized percentage increase in acceptor (1 mole % Rhod-PE-DOPC:PIP2) by 0.5 μM of donor Alexa 488 WT vs. PBD mutant ATP8b1 is plotted as bar diagram. Data plotted are mean±S.E.

FIG. 3J is an image showing Microscopic liposomes-giant unilamellar vesicle (GUVs) of DOPC:PIP2 (95:5) doped with 1 mole % of Rhodamine PE (red fluorescence) incubated with 0.5 μM of Alexa-488 labeled (green fluorescence) ATP8b1-WT and ATP8b1-PBD protein. Top panel is showing Alexa488-ATP8b1-WT on the equatorial plane of GUV vs. Alexa 488-ATP8b1-PBD mutant (lower panel).

FIG. 3K is a graph Oval profile showing quantitative analysis of interaction between GUVs with WT or PBD-mutant ATP8b1.˜ 25 GUVs were quantified for both samples and the data is presented as mean±SD, p>0.0001 with t-test.

FIG. 4A shows plots showing gene ontology terms that are significantly enriched with an adjusted P-value less than 0.05 in the differentially expressed gene sets (up to 40 terms). Significantly differentially expressed genes were clustered by their gene ontology and the enrichment of gene ontology terms was tested using Fisher exact test (GeneSCF v1.1-p2).

FIG. 4B is a volcano plot showing comparison of the global transcriptional change across the WT vs. ATP8b1^{-/-} HePG2 cells. Each data point in the scatter plot represents a gene. The log 2 fold change of each gene is represented on the x-axis and the log 10 of its adjusted p-value is on the y-axis. Genes with an adjusted p-value less than 0.05 and a log 2 fold change greater than 1 are indicated by red dots. These represent up-regulated genes. Genes with an adjusted p-value less than 0.05 and a log 2 fold change less than-1 are indicated by green dots. These represent down-regulated genes.

FIG. 5A shows THP-1 WT or ATP8b1^{-/-} macrophages (differentiated by PMA treatment) were treated with ±1 μg/ml of LPS for 3 hours±5 μM of nigericin for 1 hour. Cell extracts were prepared using MPER lysis buffer and samples were resolved on SDS-PAGE, followed by transfer to PVDF membrane. The membrane was stained with Ponceau S stain and probed with human-specific cleaved GsdmD antibody.

FIGS. 5B and 5C are graphs of B) THP-1 WT and ATP8b1^{-/-} macrophages or C) THP-1 WT and ATP8b1^{-/-} monocytes were treated with ±1 μg/ml of LPS for 3 hours±5 μM of nigericin for 1 hour, followed by ELISA for measuring IL-1β release in media, with n=3, mean±SD; **** show p<0.0001, *** show p<0.001, n.s=non-significant by ANOVA posttest.

FIG. 5D is an image showing indirect immunofluorescence for lysosomal protease Cathepsin B in THP-1 WT and ATP8b1^{-/-} macrophages. WT cells treated with Ciprofloxacin were used as a positive control for Cathepsin B release in cytoplasm.

FIG. 5E is an image showing THP-1 WT and ATP8b1^{-/-} macrophages were stained with Lysotracker Deep Red (75 nM for 2 h), followed by 3× washing with PBS and fixing. Cells were imaged using high-resolution STED microscopy.

FIG. 5F is an image of THP-1 WT and ATP8b1^{-/-} macrophages were incubated with Acridine orange (2.6 μM for 10 min), followed by washing, fixing, and imaging of cells using confocal microscopy.

FIG. 5G shows THP-1 WT or ATP8b1^{-/-} macrophages were treated with ±MCC950 (10 μM)±LPS±Nigericin. Samples were probed for cleaved Gasdermin D via western blotting using antibody specific for cleaved N-terminal fragment of GsdmD. □-actin was used as a loading control.

FIG. 5H shows THP-1 WT or ATP8b1^{-/-} monocytes were incubated with Alexa488-labeled LPS (1 μg/ml for 3 hours), followed by washing, fixing, and imaging using confocal microscopy.

FIG. 6A shows an image of THP-1 WT and ATP8b1^{-/-} macrophages were incubated with calcein-labeled apoptotic Jurkat cells for 4 hours, and efferocytosis was visualized using confocal microscopy. Apoptosis was induced in Jurkat cells by staurosporine (1 μM) treatment for 16 hours.

FIG. 6B is a graph of quantification of efferocytosis of calcein-labeled Jurkat cells, with n≥200 cells per treatment group, values are mean±SD; ** indicate p=0.0034 by T-test.

FIG. 6C is an image showing indirect immunofluorescence for efferocytosis receptor MerTK in THP-1 WT and ATP8b1^{-/-} macrophages.

FIG. 6D is an image showing THP-1 WT and ATP8b1^{-/-} macrophages were incubated with FITC-labeled latex beads 1 hour, and phagocytosis was visualized using confocal microscopy.

FIG. 7 is a schematic model showing the novel role of ATP8b1 in PIP2 flip, LPS-induced non-canonical GsdmD cleavage, and in enhancing Nlrp3 inflammasome-induced IL-1β release.

FIG. 8A is an image of WT or ATP8b1^{-/-} HepG2 cells pre-incubated with phospholipase inhibitors at 20° C. were supplemented with NBD-PIP2 followed by incubation for up to 60 min to allow lipid internalization. Cells were subjected to back-exchange with BSA, followed by TBSS wash, and visualization by confocal laser scanning microscopy. FIRE look-up table (ImageJ) was used to highlight intensity variations in PIP2 flip. Images are representative of three independent experiments. Scale bar: 20 μm.

FIG. 8B is an image of WT or ATP8b1^{-/-} HepG2 cells pre-incubated with phospholipase inhibitors at 20° C. were supplemented with NBD-PE followed by incubation for up to 60 min to allow lipid internalization. Cells were subjected to back-exchange with BSA, followed by TBSS wash, and visualization by confocal laser scanning microscopy. FIRE look-up table (ImageJ) was used to highlight intensity variations in PIP2 flip. Images are representative of three independent experiments. Scale bar: 20 μm.

FIG. 8C is a graph showing quantification of flipped NBD-PIP2 in WT and ATP8b1^{-/-} HePG2 cells (N=25 cells, values are mean±SD; **** show p<0.0001 by t-test.

FIG. 8D is a graph showing quantification of flipped NBD-PE in WT and ATP8b1^{-/-} HePG2 cells (N=25 cells, values are mean±SD; **** show p<0.0001 by t-test.

FIG. 8E is a bar graph showing ATPase activity of ATP8B1 in presence of various lipids (300 UM).

FIG. 8F is a graph showing phospholipid concentration-dependent ATPase activity of ATP8B1. Graph representing data points of three independent experiments, values are mean±SD and fitted by a non-linear dose-dependent curve.

FIG. 8G is a schematic diagram for PIP2 flip assay in ATP8B1 proteoliposomes vs. empty liposomes.

FIG. 8H is a graph showing sodium dithionite quenching of NBD-PIP2 on the outer side of the proteoliposomes vs. empty liposomes over 10 min, Triton-X-100 was used to quench all the remaining NBD-PIP2.

FIG. 8I is a graph showing the percentage PIP2 flipped over time in proteoliposomes vs. empty liposomes±sodium dithionite, ±Triton-X-100, data are from 3 independent experiments, values are mean±SD, **** show p<0.0001 by t-test.

FIG. 8J is a graph showing sodium dithionite quenching of NBD-PE on the outer side of the proteoliposomes vs. empty liposomes over 10 min, Triton-X-100 was used to quench the remaining inner leaflet NBD-PE.

FIG. 8K is a bar graph showing the percentage PE flip over time in proteoliposomes vs. empty liposomes±Sodium dithionite, ±Triton-X-100. The data are from 3 independent experiments, values are mean±SD, n.s, non-significant by t-test.

FIG. 9A is an image of a western blot gel showing THP-1 WT or ATP8B1^{-/-} monocytes were treated with ±1 μg/ml of LPS for 3 hours±5 μM of nigericin for 1 hour. Cell extracts were probed with human-specific cleaved GSDMD antibody, GAPDH serves as loading control.

FIG. 9B is a graph showing quantification of western blot bands from FIG. 9A.

FIG. 9C is a bar graph showing ELISA for measuring ILIβ release in media from THP-1 WT or ATP8B1^{-/-} monocytes, with n=3, mean±SD; **** show p<0.0001, *** show p<0.001, n.s=non-significant by ANOVA posttest.

FIG. 9D is a bar graph showing ELISA for measuring ILIβ release in media from THP-1 WT or ATP8B1^{-/-} macrophages, with n=5, mean±SD; **** show p<0.0001, n.s=non-significant by ANOVA posttest.

FIG. 9E is a schematic diagram showing BMDMs from WT and Atp8b1^{-/-} mice treated with ±LPS±Nig.

FIG. 9F is an image of a western blot of BMDMs derived from C57BL6J WT or Atp8b1^{-/-} mice were treated with ±1 μg/ml of LPS for 3 hours±5 μM of nigericin for 1 hour. Cell extracts were probed with mouse-specific cleaved GSDMD antibody, b-actin serves as loading control.

FIG. 9G is a bar graph showing ELISA for measuring IL1β release in media from WT or Atp8b1^{-/-} BMDMs, with n=3, mean±SD; ** p=0.0012 by t-test.

FIG. 9H is an image of a western blot of cleaved N-terminal fragment of GSDMD from BMDMs derived from C57BL6/J-WT or Atp8b1^{-/-} mice were treated with ±10 μg/ml of LPS for 3 hours.

FIG. 9I is an image of indirect immunofluorescence for N-terminal fragment of GSDMD in THP-1 WT and ATP8b1^{-/-} macrophages with ±1 μg/ml of LPS for 3 hours #5 μM of nigericin for 1 hour. Wheat Germ Agglutinin (WGA) is used to stain the membrane.

FIG. 9J is a bar graph showing C57BL6J WT or Atp8b1^{-/-} mice (WT n=5, ATP8b1^{-/-} n=7,) were injected intraperitoneally with a lethal dose of LPS (25 mg/kg). ELISA showing plasma IL1β post 4h LPS injection.

FIG. 9K is a survival curve for WT and Atp8b1^{-/-} mice, p=0.0033 by Log-rank test.

FIG. 10A is a graph showing the Young's modulus (E_{Y}, Pa) representing stiffness of WT and ATP8B1^{-/-} cells. In all cases, data is from 3 replicates, with 323 □n□493 cells for E_{Y} values, 182□n□561 cells for F_{ad} values, and 64 □n□201 for F_{T}, T_{M} and R_{T} values. Circles represent the data points and lines represent the mean±S.E. * p<0.01, ** p<0.001, **** p<0.0001.

FIG. 10B is a graph showing the Young's modulus (E_{γ}, Pa) representing stiffness of WT±PIP2 and ATP8B1^{-/-} cells±PIP2.

FIG. 10C is a graph showing the adhesive forces (F_{ad}) between the AFM tip and the cell surface for WT and ATP8B1^{-/-} cells.

FIG. 10D is a graph showing the adhesive forces (F_{ad}) between the AFM tip and the cell surface for WT±PIP2 and ATP8B1^{-/-} cells±PIP2.

FIG. 10E is a graph showing membrane tether forces (F_{T}), for WT and ATP8B1^{-/-} cells.

FIG. 10F is a graph showing membrane tether forces (F_{T}), for WT±PIP2 and ATP8B1^{-/-} cells±PIP2.

FIG. 10G is a graph showing the apparent plasma membrane tension (T_{M}) noted for WT and ATP8B1^{-/-} cells (based on F_{T} values).

FIG. 10H is a graph showing the apparent plasma membrane tension (T_{M}) noted for WT±PIP2 and ATP8B1^{-/-} cells±PIP2 (based on F_{T} values).

FIG. 10I is a graph showing the tether radius (R_{T}), obtained from the F_{T} values, for WT and ATP8B1^{-/-} cells. In all cases, data is from 3 replicates, with 323 □n□493 cells for E_{Y} values, 182≤n≤561 cells for F_{ad} values, and 64≤n≤201 for F_{T}, T_{M} and R_{T} values. Circles represent the data points and lines represent the mean±S.E. * p<0.01, ** p<0.001, **** p<0.0001.

FIG. 10J is a graph showing the tether radius (R_{T}), obtained from the F_{T} values, for WT±PIP2 and ATP8B1^{-/-} cells±PIP2.

FIG. 10K is a schematic diagram depicting intercalating of charge-shift probe Di-8-ANEPPS to the cell membrane and excitation spectra showing changes in the orientation of the probe in the membrane bilayer.

FIG. 10L is a graph showing dipole potential of the PM in various WT vs. ATP8B1^{-/-} cells. Bar graphs showing independent triplicates, data is mean±S.E, ** p<0.01, *** p<0.001, **** p<0.0001 by t-test.

FIG. 10M is an image showing representative STEDYCON micrograph showing Di-8-ANEPPS labeled RAW 264.7 WT and Atp8b1^{-/-} cells (Ext: 460 nm, left panel, Ext: 561 nm middle panel, and the emission bandpass was fixed at 670 nm). The 3D intensity plot corresponding to the fluorescence intensity ratio R (color-coded) was calculated using ImageJ.

FIG. 10N is a graph showing dipole potential of WT RAW 264.7 compared with Atp8b1^{-/-} cells±supplementation with 5 μM and 10 μM PIP2 liposomes, the bar graph is showing values with mean±S.E. data is mean±S.E, *** p<0.001, **** p<0.0001, n.s.=non-significant by t-test.

FIG. 11A is a bar graph showing plasma IL1b values in Atp8b1^{-/-} mice injected with LPS vs. WT mice.

FIG. 11B is a graph showing mean survival time for Atp8b1^{-/-} mice vs. WT mice.

### DETAILED DESCRIPTION

Various technologies pertaining to a composition, system, and method for treating inflammatory manifestations in PFIC1 patients and related conditions, are described herein. The general inventive concepts provide a method to use GsdmD inhibitors for alleviation of inflammatory manifestations in PFIC1 patients to treat one or more conditions or diseases, or symptoms thereof, described herein.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

As used herein, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any combination of method or process steps as used herein may be performed in any order, unless otherwise specified or clearly implied to the contrary by the context in which the referenced combination is made.

The terms “susceptible” and “at risk” as used herein, unless otherwise specified, mean having little resistance to a certain condition or disease relative to the general population, including being genetically predisposed, having a family history of, and/or having symptoms of the condition or disease. The term refers to those having a vulnerability higher than the general population.

The terms “modulating” or “modulation” or “modulate” as used herein, unless otherwise specified, refer to the targeted movement of a selected characteristic.

The term “ameliorate” as used herein, unless otherwise specified, means to eliminate, delay, or reduce the prevalence or severity of symptoms associated with a condition.

The term “an effective amount” is intended to qualify the amount of a composition according to the general inventive concepts which will achieve the goal of decreasing the risk that the individual will suffer an adverse health event (e.g., cardiovascular event related to high cholesterol), including reducing one or more symptoms, while avoiding adverse side effects such as those typically associated with alternative therapies. The effective amount may be administered in one or more doses.

The terms “treating”, and “treatment” as used herein, unless otherwise specified, includes delaying the onset of a condition, reducing the severity of symptoms of a condition, or eliminating some or all of the symptoms of a condition.

In certain embodiments, the general inventive concepts contemplate compositions and methods for reducing blood lipid concentration. In certain embodiments, the general inventive concepts contemplate compositions and methods for modulating expression of one or more cholesterol efflux pathways. This, in turn, leads to a reduction in cholesterol in an individual.

The general inventive concepts are based, in part, on the discovery that ATP8b1, a human disease-causing gene, plays a role in maintaining PIP2 on the inner leaflet of PM and in flipping exogenous PIP2, making it the first known PIP2 flippase from any system. PFIC1 patients often show extrahepatic inflammatory symptoms and disorders of fat/cholesterol processing (e.g., fat malabsorption presenting as fatty diarrhea and excess cholesterol release in feces), but the mechanism of these manifestations is not clear. We describe a novel role of ATP8b1 in regulating GsdmD cleavage via non-canonical inflammasome pathway. Humans are always exposed to small doses of LPS (from gut bacteria), Applicants believe this low-dose LPS exposure may fuel inflammasome activity in ATP8b1^{-/-} immune cells. Our data provide evidence for use of GsdmD inhibitors for alleviation of inflammatory manifestations in PFIC1 patients.

Human mutations in ATP8b1 (a member of the type 4 subfamily of P-type ATPases) can cause progressive familial intrahepatic cholestasis type (PFIC1), characterized by enhanced biliary cholesterol excretion and increased extrahepatic inflammation. The hepatic canalicular membrane is highly enriched in cholesterol and sphingomyelin, allowing formation of rigid membrane, that serve as a strong barrier to bile salt-mediated lipid extraction. Mutations in ATP8b1 perturb the detergent-resistant state of the hepatic canalicular membrane, leading to excessive cholesterol extraction by bile salts and hepatic injury. The loss of cholesterol from canalicular membrane in PFICl is proposed to negatively regulate the activity of the bile salt exporter (BSEP), leading to increased cholestasis. While much is known about the pathophysiology of PFIC, the underlying mechanisms for how the loss of ATP8b1 leads to compromised canalicular membrane integrity and extrahepatic inflammation are not clear.

In addition to PFIC1, ATP8b1 also plays a role in various disease conditions such as diabetes, inflammation, neural degeneration, as well as in physiological processes such as normal hearing. These data indicate involvement of ATP8b1 in maintaining general cellular homeostasis. While not wishing to be bound by any theory, ATP8b1 is proposed to maintain the plasma membrane (PM) asymmetry by translocation of phospholipids from the outer to the inner leaflet of the PM. The phosphatidylserine (PS) flippase activity of ATP8b1 was proposed to be involved in maintaining membrane integrity, but other studies have shown the role of ATP11C in PS flip and ATP8b1 in phosphatidylcholine (PC) flip. Another study proposed a flippase-independent function, where ATP8b1 affected the formation of the microvilli structures. Interestingly, a high throughput proteomic analysis proposed that ATP8b1 flippase activity and phosphoinositide metabolism may be interconnected at the Golgi, and more recently ATP8b1 was shown to be regulated by phosphoinositides, but the mechanistic link between ATP8b1 and phosphatidylinositol phosphates (PIPs) metabolism is not clear. The most commonly studied PIP species include phosphatidylinositol-4,5-bisphosphate (PIP2), which plays a role in many cellular processes such as cell signaling. ATP8b1 plays a physiological role in hearing, as ATP8b1 mutation causes hearing loss associated with progressive degeneration of cochlear hair cells². Similar to ATP8b1, PIP2 is also critical for mechanical transduction and adaptation of hair cells.

Human PFIC1 patients often present with extra-hepatic inflammatory clinical features, such as steatohepatitis, fat malabsorption, diarrhea, pancreatitis, and atherosclerosis. Even after liver transplantation, which rescues hepatic symptoms, the extrahepatic manifestations of PFIC1 persists. These data indicate that the role of ATP8b1 in inflammation may be independent of cholestasis. ATP8b1 deficiency in human peripheral blood monocyte-derived macrophages (HMDMs) was shown to result in incomplete polarization of HMDMs into M2c (subset of alternatively activated macrophages that are involved in suppression of immune responses). Recent studies have highlighted the major role of pyroptosis executor Gasdermin D (GsdmD), a pore-forming protein, in promoting a variety of inflammatory diseases. Pyroptosis, induced by GsdmD mediated membrane pore formation and ensuing cell swelling, can be caused by canonical inflammasome via cleavage of caspase-1 through Nlrp3 or via Nlrp3-independent non-canonical pathway. Active caspase-1 or 11 cleaves GsdmD, and the newly formed N-terminal fragments of GsdmD (NT-GsdmD) can activate canonical inflammasome in a positive feedback loop. GsdmD is thus a common executor of both canonical and non-canonical inflammasome activity, and GsdmD cleaved via non-canonical pathway can induce Nlrp3 inflammasome activity. The central theme is that GsdmD pores on the plasma membrane allow the release of inflammatory interleukins (IL-1β, IL-18), and other inflammation-promoting molecules, amplifying the inflammatory cascade and inducing disease progression.

Here, we determine the role of ATP8b1 in PIP2 trafficking and inflammasome activity. Using Crispr-Cas9 generated homozygous ATP8b1 knockouts (ATP8b1^{-/-}) in mouse (RAW264.7 cells), human monocytes (THP-1 cells), human macrophages (differentiated THP-1 cells), human hepatocytes (HepG2), and human embryonic kidney (HEK293) cells, we tested if ATP8b1 is involved in restricting PIP2 at the inner leaflet of the plasma membrane. Moreover, we determined if ATP8b1 is specifically involved in flipping exogenous PIP2. The role and mechanism of ATP8b1 in regulation of inflammasome-mediated cleavage of GsdmD and in inflammation-resolving efferocytic pathway was determined. Efferocytosis serves as a major anti-inflammatory mechanism, with a prime example being it's athero-protective role in regression of plaques. Finally, the underlying mechanisms for LPS-induced GsdmD cleavage in ATP8b1^{-/-} cells were determined.

Recent studies highlight an intricate cross-talk between ATP8b1 and phosphoinositide metabolism, but the mechanistic details of the interplay between ATP8b1 and phosphatidylinositol phosphates (PIPs) metabolism is not clear.

In certain embodiments, the general inventive concepts contemplate a method of treating, preventing, inhibiting, or ameliorating the symptoms of progressive familial intrahepatic cholestasis 1 (PFIC1) in an individual in need thereof, the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

In certain embodiments, the general inventive concepts contemplate a method of treating, preventing, inhibiting, or ameliorating hepatocyte damage in an individual in need thereof, the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

In certain embodiments, the general inventive concepts contemplate a method of reducing inflammation in an individual suffering from symptoms of progressive familial intrahepatic cholestasis 1 (PFIC1), the method comprising administering a therapeutically effective amount of at least one of a GsdmD blocker, a PIP2 inhibitor, and an antisense oligonucleotide to the individual in need thereof. In certain exemplary embodiments, the GsdmD blocker is selected from the group comprising: disulfiram and dimethyl fumarate. In certain exemplary embodiments, the PIP2 inhibitor is selected from the group comprising: ISA2011b, IC-87114, BKM120, CAL-101, and LY 294002. In certain exemplary embodiments, the antisense oligonucleotide against e.g., the PIP2 biosynthetic enzyme PIP5K1a.

Disulfiram (also known as Antabuse), an FDA-approved drug for alcoholism, was shown to bind directly to the cleaved N-terminal fragment of GsdmD (The paper was published by a group at Harvard Medical School and appeared in Nature Immunol. 2020 May 4; 21 (7): 736-745 and has already been cited over 800 times. At nanomolar concentration, disulfiram covalently modifies human/mouse Cys191/Cys192 in GSDMD to block pore formation. Authors showed that Disulfiram still allows IL-1β and GSDMD processing, but abrogates pore formation, thereby preventing IL-1β release and pyroptosis. Another inhibitor is dimethyl fumarate (DMF) that promotes succination of GsdmD. GSDMD succination prevents its interaction with caspases, limiting its processing, oligomerization, and capacity to induce cell death. These data were published in journal “Science” 2020 Sep. 25; 369 (6511): 1633-1637) and has been cited over 500 times.

Also described is a method wherein the disease or disorder is one or more of inflammation, extra-hepatic inflammatory clinical features, such as steatohepatitis, fat malabsorption, pancreatitis, sporadic hearing loss, Alzheimer's disease, diarrhea, pancreatitis, and atherosclerosis.

In accordance with the methods of the present invention, a GsdmD blocker may be administered to the individual in need thereof for a time period of at least 2 days, or at least 3 days, or at least 5 days, or at least 6 days, or at least 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 14 weeks, or at least 16 weeks, or at least 18 weeks, or at least 24 weeks or longer. In specific embodiments of the methods, a GsdmD blocker is administered to an individual once or multiple times daily or weekly. In specific embodiments, GsdmD blocker is administered to the subject from about 1 to about 6 times per day or per week, or from about 1 to about 5 times per day or per week, or from about 1 to about 4 times per day or per week, or from about 1 to about 3 times per day or per week. In other specific embodiments, the GsdmD blocker is administered once or twice daily for a period of at least 2 days, at least 3 days, at least 4 days, at least 5 days or at least 6 days, or at least one week, at least two weeks, at least three weeks, at least four weeks, at least five weeks, or at least six weeks.

In accordance with the methods of the present invention, a PIP2 inhibitor may be administered to the individual in need thereof for a time period of at least 2 days, or at least 3 days, or at least 5 days, or at least 6 days, or at least 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 14 weeks, or at least 16 weeks, or at least 18 weeks, or at least 24 weeks or longer. In specific embodiments of the methods, a PIP2 inhibitor is administered to an individual once or multiple times daily or weekly. In specific embodiments, PIP2 inhibitor is administered to the subject from about 1 to about 6 times per day or per week, or from about 1 to about 5 times per day or per week, or from about 1 to about 4 times per day or per week, or from about 1 to about 3 times per day or per week. In other specific embodiments, the PIP2 inhibitor is administered once or twice daily for a period of at least 2 days, at least 3 days, at least 4 days, at least 5 days or at least 6 days, or at least one week, at least two weeks, at least three weeks, at least four weeks, at least five weeks, or at least six weeks.

In certain embodiments In certain embodiments of the methods of the invention, the individual in need thereof is administered a therapeutically effective amount of a GsdmD blocker or a PIP2 inhibitor. This amount will vary depending on the individual and the specific therapeutic endpoint.

In certain embodiments, the general inventive concepts contemplate compositions and methods for reducing symptoms of progressive familial intrahepatic cholestasis type 1 (PFIC1) by blocking GsdmD and thereby reducing extrahepatic inflammation. In certain embodiments, the general inventive concepts contemplate compositions and methods for modulating expression of inflammatory pathways associated with GsdmD. This, in turn leads to a reduction in excessive cholesterol extraction by bile salts in an individual.

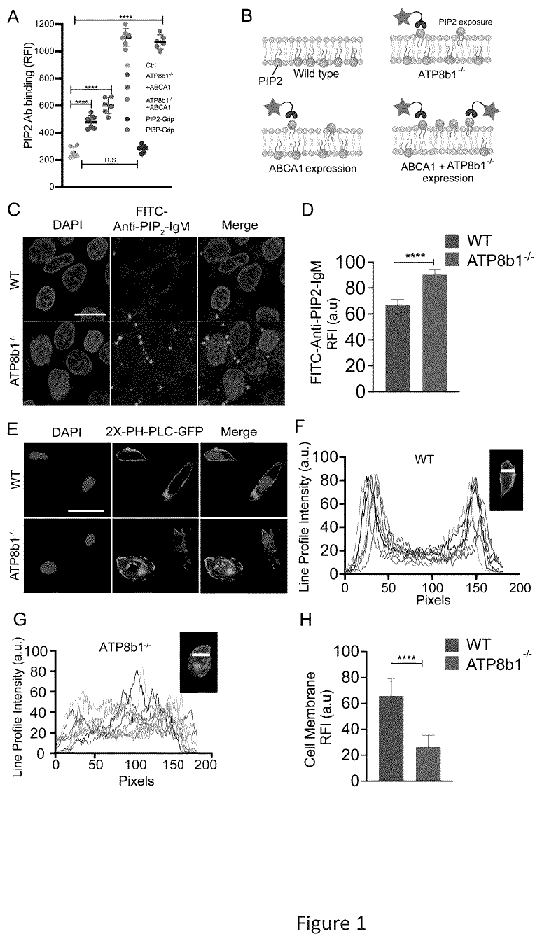

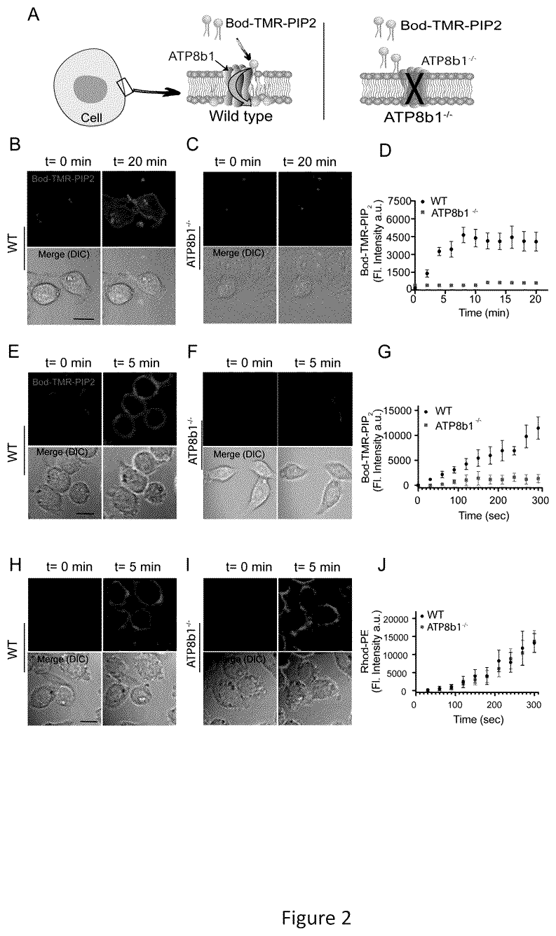

Using homozygous ATP8b1^{-/-} in variety of cell types, we show that PIP2 is exposed at the cell-surface of cells lacking ATP8b1. PIP2 exposure was further augmented in ATP8b1^{-/-} cells expressing ABCA1 (FIG. 1), indicating that ATP8b1 and ABCA1 are involved in flip-flop of PIP2, and cells may co-regulate expression of these genes to maintain appropriate PIP2 levels across PM. In support of this argument, previous studies have shown co-regulation of ATP8b1 and ABCA1 by microRNA MiR-33. Increased PIP2 exposure does not necessarily prove that PIP2 is depleted from the inner leaflet of PM lipid bilayer. Thus, we used the pleckstrin-homology (PH) domain of phospholipase C (2X-PH-PLC), a highly specific reporter that binds only PIP2, and not to other close PIP species such as PI-3,4-P2 or PI-3,4-P2. We found that the exposed PIP2 at the cell-surface in ATP8b1^{-/-} cells is accompanied by parallel reduction in PIP2 levels from the inner leaflet of the PM. Taken together, these data provided strong, but still indirect, evidence that ATP8b1 is involved in PIP2 flip. To directly assess the role of ATP8b1 in translocating PIP2 from the outer leaflet of the PM to the inner leaflet, we standardized a time-lapse live cell imaging microscopy-based assay using exogenous Bodipy-TMR labeled PIP2. Boron dipyrromethene (BODIPY) is a highly lipophilic neutral fluorophore that emits fluorescence only when embedded within hydrophobic lipid environment. Thus, bodipy-PIP2 remains non-fluorescent in media until it latches and gets inserted in plasma membrane lipid bilayer. In WT cells, the bodipy-PIP2 binds to the outer layer and is then translocated to the inner leaflet by ATP8b1, leading to strong bodipy signal at the plasma membrane. The ATP8b1^{-/-} cells, which are defective in flipping PIP2, bodipy-PIP2 does transiently bind to outer surface of cell-surface but get exchanged back to the media, leading to weak bodipy-PIP2 signal at the PM (FIG. 2). Interestingly, the kinetics of PIP2 flip is cell-type dependent, with macrophages showing much faster PIP2 flip vs. hepatocytes (FIG. 2), indicating that PIP2 may be flipped at varied rates across different tissues. The physiological relevance of differential kinetics is not clear, but, while not wishing to be bound by any theory, we believe that cells involved in PIP2-mediated signaling pathways may regulate the rate of PIP2 flip to modulate signaling events. The redistribution of PIP2 from the inner leaflet of the PM to the cell-surface can have several physiological consequences. For example, we have shown before that exposed PIP2 promotes lipid solubilization by cholesterol acceptors such as apoA1, leading to increased cholesterol extraction. Bile salts, similar to apoA1, serve as cholesterol acceptors with ABCA1 playing role in cholesterol efflux to both apoA1 and bile salts. Thus, increased PIP2 exposure can reduce detergent-resistant property of membrane, enhancing cholesterol extraction and hepatocyte damage.

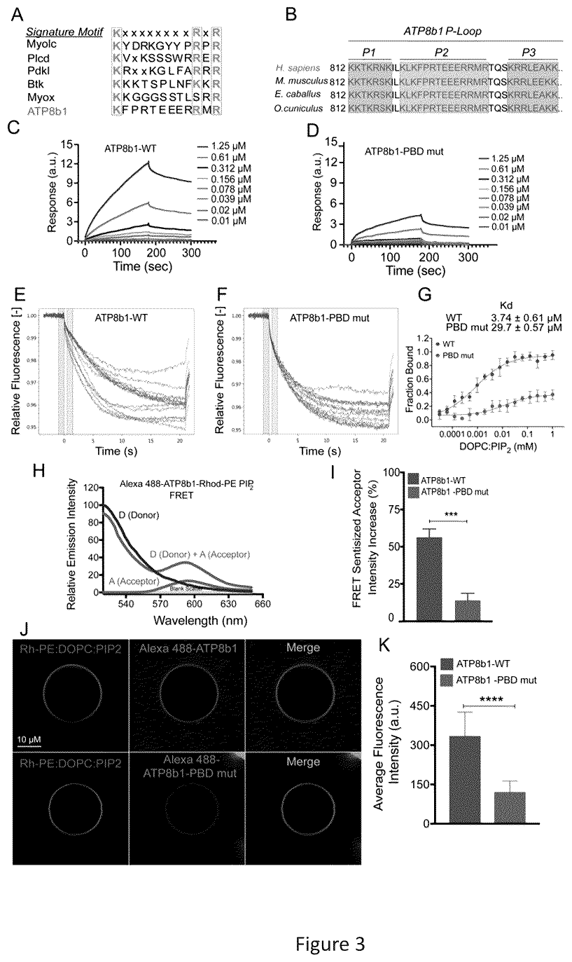

ATP8b1 also plays a physiological role in hearing, as ATP8b1 mutation causes hearing loss, associated with progressive degeneration of cochlear hair cells. Interestingly, similar to ATP8b1, PIP2 is also critical for mechanical transduction and adaptation of hair cells, indicating the intricate link between PIP2 and the physiological functions of ATP8b1. The cross talk between ATP8b1 and PIP2 trafficking/metabolism was further strengthened by our data showing that ATP8b1 contains a PIP2 binding domain, which allows direct binding between ATP8b1 and PIP2 (FIG. 3). Using computational analysis, we identified a conserved and putative PIP2 binding motif KFPRTEEERRMR, from amino acids 824-835 in P2-loop of the phosphatase (P) domain of ATP8b1, and this domain is not present in other related ATPase family members. We used a variety of biophysical assays using immobilized as well as solubilized protein fragments to show direct binding of PIP2 with ATP8b1. The significance of PIP2 binding region of ATP8b1 was highlighted by diminished PIP2 binding observed with PBD mutant isoform of ATP8b1 (FIG. 3). Mutations in the PIP2 binding domain of ATP8b1, such as R833W, are found in human patients, but the physiological role of this domain is not yet described. Further studies are required to decipher the physiological functions of the ATP8b1-PIP2 interaction. We believe that PIP2 binding serves as a regulatory mechanism for modulating ATP8b1 activity. For example, under conditions of increased PIP2 at the inner leaflet of plasma membrane, cells may block PIP2 flip, so that PIP2 can stay exposed and can get effluxed out of the cell along with cholesterol via apoA1-ABCA1 pathway. We have previously shown that exposed PIP2 promotes lipid solubilization by cholesterol acceptors and enhance cholesterol efflux. Thus, PIP2 binding may regulate ATP8b1 protein levels or activity to counter perturbations in lipid homeostasis via modulating protein degradation or protein trafficking pathways.

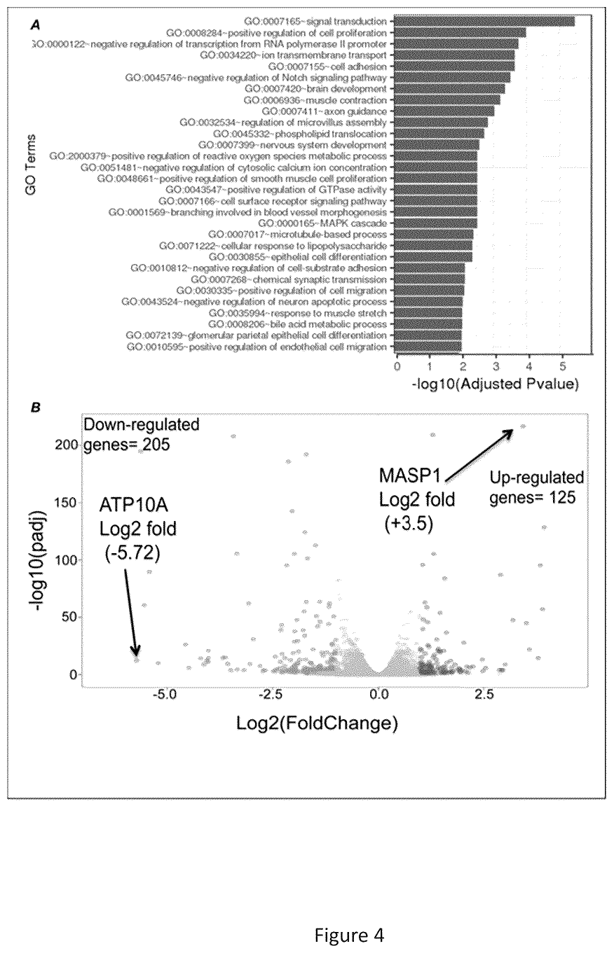

For unbiased insights on the global transcriptome alterations in cells lacking ATP8b1, RNAseq analysis of ATP8b1^{-/-} vs. WT cells was performed. The top altered pathways in ATP8b1^{-/-} vs. WT hepatocytes included signal transduction, cell-adhesion, microvillus assembly, phospholipid translocation, response to LPS, and bile acid metabolism (FIG. 4). Importantly, PIP2 plays a role in signal transduction, microvillus assembly, cell-adhesion, and migration. Top altered genes in ATP8b1^{-/-} cells included MASP1 (part of complement pathway, promotes inflammation) and ATP10A (regulated by cholesterol). These data indicate cross talk between ATP8b1, PIP2, and cholesterol/bile-acid metabolism (FIG. 4). These interactions may play a role in fine-tuning cellular signaling and inflammatory responses.

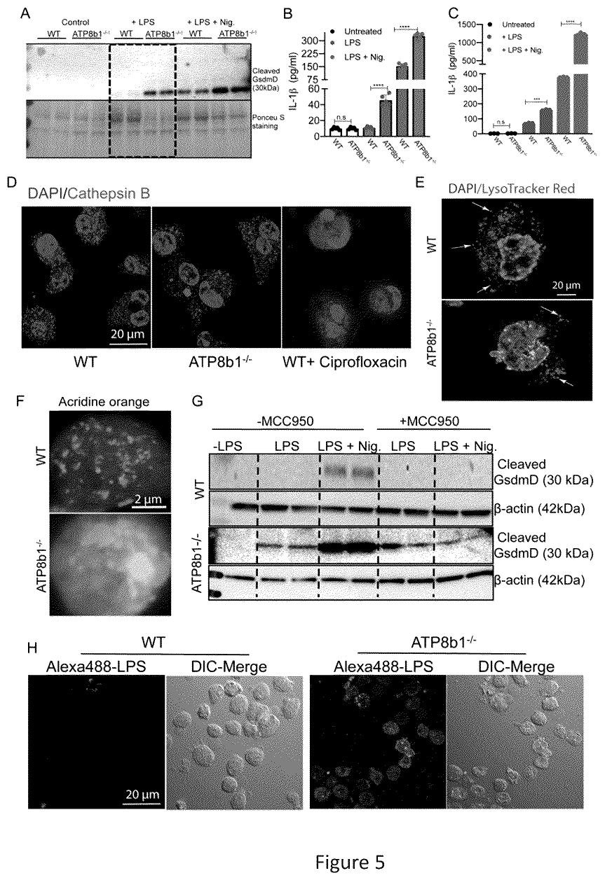

Though ATP8b1 deficiency is primarily characterized by cholestasis, PFIC1 patients also show multiple extrahepatic inflammatory manifestations such as secretory diarrhea, steatohepatitis, and pancreatitis. Interestingly, these disease features are independent of cholestasis, as these inflammatory conditions persist even after liver transplant. These data indicate that ATP8b1 may be involved in regulating inflammatory activity of immune cells. ATP8b1 was shown to regulate the polarization of human monocyte derived macrophages (HMDMs) into M2c (anti-inflammatory macrophages). Recent studies have highlighted the major role of pyroptosis executor GsdmD in promoting a variety of inflammatory diseases. Activity of both canonical and non-canonical inflammasome pathways result in GsdmD mediated membrane pore-formation and release of pro-inflammatory cytokines such as IL-1β/IL-18. GsdmD can be cleaved by Nlrp3 mediated canonical inflammasome pathway, where LPS serves as priming signal, while high levels of extracellular ATP, or compromised lysosomal membrane integrity serves as a secondary signal. Strikingly, GsdmD was found to be cleaved in ATP8b1^{-/-} monocytes as well as macrophages upon exposure to LPS alone (FIG. 5). Thus, we tested if this was due to preexistence of secondary signal within the ATP8b1^{-/-} cells. No changes were observed in basal or LPS induced nuclear translocation of NF-kB, ruling out the possibility of increased transcription of pro-IL-1β. We did observe reduced lysotracker staining in ATP8b1^{-/-} macrophages (FIG. 5) and tested if this is due to dampened lysosomal biogenesis or stimulated lysosomal disintegration. No differences were observed in basal or starvation-induced nuclear translocation of TFEB, a master regulator of lysosomal biogenesis. There was no release of Cathepsin B from lysosomes to the cytoplasm in ATP8b1^{-/-} cells (FIG. 5), indicating that integrity of lysosomal membrane was also maintained. Given that number or membrane integrity of lysosomes seems normal, we probed the lysosomal pH in ATP8b1^{-/-} cell, as the normal acidic pH is required for lysosomal staining with lysotracker dye. Using acridine orange, we found lysosomal pH to be less acidic in ATP8b1^{-/-} cells. The acidic pH of lysosomes is maintained via active pumping of H⁺ ions from the cytosol across the lysosomal membrane by V-type ATPases, thus ATP8b1 may have a direct or indirect role in regulating expression/function of these proteins.

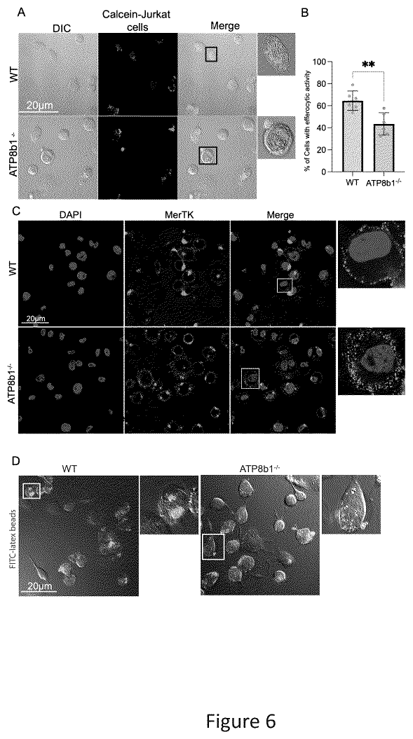

We deciphered the mechanism of LPS induced GsdmD cleavage by showing that LPS can gain entry into the ATP8b1^{-/-} cells, leading to Nlrp3 independent and non-canonical inflammasome dependent GsdmD cleavage (FIG. 5). The final readout of inflammasome activity and GsdmD activity is release of mature IL-1β and we found markedly higher IL-1 β release in both monocytes and macrophages upon LPS or LPS+Nigericin treatments (FIG. 6). Increased IL-1 β release from ATP8b1^{-/-} macrophages could be due to GsdmD cleavage upon LPS exposure, leading to increased pore formation for IL-1□□release upon induction of canonical inflammasome pathway. Another possibility is that non-canonical GsdmD cleavage can subsequently activate the canonical Nlrp3 inflammasome pathway, leading to increased cleavage of IL-1 β and subsequently increased IL-1 β release via GsdmD pores. Recent studies have highlighted the role of efferocytosis and phagocytosis in a growing list of chronic inflammatory diseases. Efferocytic activity entails clearance of apoptotic and damaged cells by macrophages, and efferocytosis is vital for resolution of inflammation. The macrophages lacking ATP8b1 showed marked defects in efferocytosis/phagocytosis, though the binding of latex beads or apoptotic Jurkat cells was not impaired (FIG. 6). Surprisingly, the cell-surface expression of efferocytic receptor MerTK was found to be higher in ATP8b1^{-/-} macrophages. The up-regulated MerTK expression in ATP8b1^{-/-} macrophages may be a compensatory mechanism to counter defective efferocytosis, but this doesn't seem to be enough to restore efferocytic ability of ATP8b1^{-/-} macrophages. Defective efferocytic activity may serve as another contributory factor for persistent chronic inflammation in PFIC1 patients carrying ATP8b1 mutations.

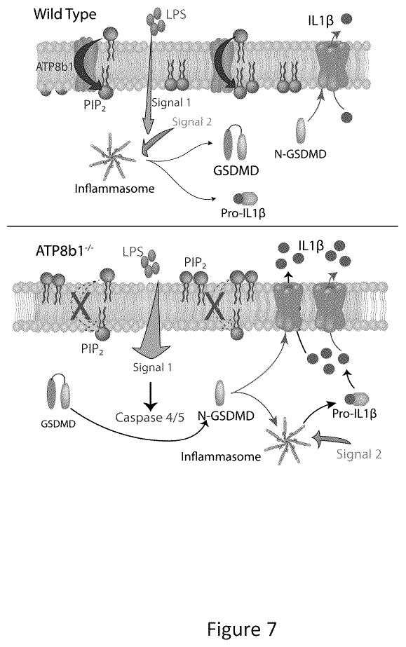

Taken together, we show that ATP8b1 is required for sequestering PIP2 at the inner leaflet of the PM and for flipping exogenous PIP2 into the PM. ATP8b1 negatively regulate GsdmD cleavage and IL-1β release from macrophages and monocytes via non-canonical inflammasome pathway. Mechanistically, cytoplasmic access of LPS in ATP8b1^{-/-} macrophages results in GsdmD cleavage in the absence of external secondary stimuli. Taken together, our data identifies ATP8b1 as a first reported PIP2 flippase from any system and as a novel negative regulator of GsdmD cleavage (FIG. 7).

The following examples illustrate features and/or advantages of the compositions, systems, and methods according to the general inventive concepts. The examples are given solely for the purpose of illustration and are not to be construed as limitations of the general inventive concepts, as many variations thereof are possible without departing from the spirit and scope of the general inventive concepts.

### Examples

ATP8b1 regulate PIP2 localization at the inner leaflet of the plasma membrane. To determine the role of ATP8b1 in PIP2 trafficking, the Crispr-Cas9 generated homozygous ATP8b1 knockouts (ATP8b1^{-/-}) in variety of cell lines were used. The knockout deletions were confirmed by sequencing and qRT-PCR. Using a flow cytometry based PIP2 antibody binding assay for live and non-permeabilized murine macrophages, we found ˜2-fold increase in cell-surface PIP2 exposure in the ATP8b1 knockout (ATP8b1^{-/-}) cells (FIG. 1A), and this data was also confirmed via fluorescent microscopy. ABCA1 expression is known to increase cell-surface PIP2 via flopping of PIP2, and an additive effect on PIP2 exposure was observed in the ATP8b1^{-/-} cells expressing ABCA1, with ˜3.6-fold increase vs. control cells (FIG. 1A, 1B). To ensure the specificity of assay, cells were pretreated with PIP2 or PI3P blocking proteins, and as shown in FIG. 1A, blocking PIP2 but not PI3P, reduced PIP2 antibody binding to the basal levels. No differences were found in binding of control IgM-FITC in WT vs. ATP8b1^{-/-} cells. These data were also confirmed in HepG2 ATP8b1^{-/-} cells via fluorescent spectroscopy, showing more PIP2 antibody binding in ATP8b1^{-/-} cells (FIG. 1C, FIG. 1D). The mechanism by which cell-surface PIP2 levels are increased in ATP8b1^{-/-} cells is not clear. To determine if the increased cell-surface PIP2 levels in ATP8b1^{-/-} cells result in reduction of PIP2 at the inner leaflet of the plasma membrane (PM) bilayer, the HepG2 cells (WT or ATP8b1^{-/-}) were stably transfected with a fluorescent PIP2 binding reporter (2X-PH-PLC-eGFP, Addgene #35142) construct. As expected, PIP2 was highly enriched at the inner leaflet of the PM in the WT cells, while the ATP8b1^{-/-} cells showed redistribution of PIP2 from PM to the cytoplasmic region (FIG. 1E), indicating reduction in PIP2 levels at the inner leaflet of the PM. The fluorescent intensity cell scan of WT cells showed two distinct fluorescent peaks originating from the two flanks of PM (FIG. 1F) while the ATP8B1^{-/-} cells showed fluorescent peak at the cytoplasmic region (FIG. 1G). Quantification of PM GFP signal showed ˜65% PIP2 is at the PM of WT cells (FIG. 1H). In contrast, with only ˜27% PIP2 at the PM (FIG. 1I). These data indicated that ATP8b1^{-/-} cells have markedly reduced PIP2 on the inner leaflet of the PM, leading to the redistribution of PIP2 reporter to the cytoplasmic region. Taken together, ATP8B1^{-/-} cells show increased PIP2 at the outer leaflet of PM and reduced PIP2 at the inner leaflet of PM. ATP8b1 flips exogenous PIP2. The increased cell-surface PIP2 and the reduced inner leaflet PIP2 levels at the PM of ATP8b1^{-/-} cells indicated that ATP8b1 may be involved in flipping of PIP2 from the cell-surface to the inner leaflet of the PM bilayer. To determine if ATP8b1 plays a direct role in translocation of PIP2 from outer leaflet of the PM, we designed a live cell time-lapse microscopy based PIP2 flip assay using fluorescent fatty acid-labeled full-length Bodipy-TMR-PIP2 (FIG. 2A) The WT HepG2 cells showed flip of exogenous PIP2 allowing it to accumulate at the PM over time, with PIP2 signal on PM appearing in ˜3-5 min (FIG. 2B), while the ATP8b1^{-/-} cells showed marked reduction in PIP2 flip and PIP2 accumulation at PM, presumably due to PIP2 exchanging back to the media (FIG. 2C). Quantification of the Bodipy-PIP2 signal at the PM over time showed robust PIP2 flip in WT cells, while the ATP8b1^{-/-} cells showed markedly defective PIP2 flip (FIG. 2D).

Similar to hepatocytes, ATP8b1 also played a role in flipping of PIP2 in macrophages, where WT macrophages showed a robust PIP2 flip with a strong bodipy-PIP2 signal at the PM within ˜30-90 seconds (FIG. 2E), indicating that kinetics of PIP2 flip is cell-type dependent. In contrast to WT macrophages, ATP8b1^{-/-} macrophages showed markedly reduced PIP2 flip (FIG. 2F). Quantification of PIP2 signal at the PM over time showed markedly defective PIP2 flip in ATP8b1^{-/-} cells vs. WT cells (FIG. 2G), while no differences were found in flip of Rhodamine-PE over time (FIG. 2H, FIG. 2I, FIG. 2J), indicating specificity of ATP8b1 toward flipping PIP2. Taken together, ATP8b1^{-/-} cells have more PIP2 on the outer leaflet of PM, lower PIP2 levels at the inner leaflet of the PM and exhibit markedly reduced flip of exogenous PIP2.

ATP8b1 directly binds PIP2. Several PIP2 binding proteins use the motif KXXXXXXXXK/RXR for binding PIP2. Using computational analysis, we identified a conserved and putative PIP2 binding motif KFPRTEEERRMR, from amino acids 824-835 in the P2 domain of P-loop of ATP8b1 (FIG. 3A, FIG. 3B). Using PONDER prediction, a helical wheel diagram, and Alpha fold prediction, we found that the PIP2 binding region of ATP8b1 is disordered and forms a putative binding surface for anionic molecules. To determine direct binding between ATP8b1 and PIP2, we expressed and purified recombinant fragment of WT or PIP2-binding-domain (PBD) mutant isoform of ATP8b1 (77 amino acid residues from 803-880). The mutated isoform contained alanine substitutions of conserved amino acid residues K824A, R832A, R833A, and R835A. The protein fragments purity and sequence were confirmed by reverse phase chromatography and Mass-Spec (LC-MS) analysis. The binding between ATP81 and PIP2 was determined using Surface Plasmon Resonance (SPR) assay, as described earlier. We immobilized WT or PBD mutant ATP8b1 by covalent coupling on a CM5 sensor chip (GE Healthcare) using EDC-NHS reagent, followed by injection of different concentrations of PIP2. Corrected response data was fitted to calculate K_{d} values. As shown in FIG. 3C, WT ATP8b1 showed robust binding with PIP2 with K_{d} of ˜11.5 μM, while the PBD mutant of ATP8b1 showed weak binding with K_{d} of ˜63.6 μM (FIG. 3D). To ensure that interaction between ATP81 and PIP2 is not limited to immobilized protein and can occur in solution form, we performed Micro Scale Thermophoresis (MST), Fluorescence Resonance Energy Transfer (FRET), and Giant Unilammellar Vesicles (GUVs)-protein interaction assays. The WT or PBD mutant ATP8b1 protein fragments were labeled with Alexa-488. MST is an immobilization-free technology for measuring interactions between biomolecules and was used to detect direct binding between PIP2 and ATP8b1. Using 1,2-Dioleoyl-sn-glycero-3-phosphocholine (DOPC)±PIP2 liposomes (lipid mole ratio 0.95:0.5) with concentrations ranging from 30 nM to 1 mM were mixed with Alexa 488-labeled WT or PBD mutant of ATP8b1. A dose-dependent alteration in mobility of WT ATP8b1 protein on the surface of PIP2 containing liposomes was observed (FIG. 3E), indicating strong interaction between ATP8b1 and PIP2, while PBD mutant of ATP8b1 showed markedly weak interaction (FIG. 3F). The K_{d} of WT ATP8b1 was ˜3.74±0.61 μM, while the PBD-mutant of ATP8b1 showed a binding with K_{d} of ˜29.7±0.57 μM (FIG. 3G). To determine if ATP8b1 and PIP2 are in close proximity (<10 nm) and interact in membrane environment, a spectroscopy-based FRET was performed. A clear interaction was observed between ATP8b1 and PIP2 (FIG. 3H), while PBD mutant of ATP8b1 showed much lower FRET efficiency (FIG. 3I). To determine interaction between PIP2 and ATP8b1 in micrometer scale, we performed confocal microscopy using giant unilamellar vesicles (GUVs). As shown in FIG. 3J, a strong binding of ATP8b1 was observed on the membrane surface of PIP2-containing GUVs, with a homogenous distribution of Alexa-488-WT-ATP8b1 (green fluorescence) on equatorial section of GUVs (red fluorescence). The PBD mutant isoform of ATP8b1 showed markedly reduced binding with PIP2-containing GUVs (FIG. 3J). Quantification of oval profile of Alexa488 revealed significantly higher binding of WT-ATP8b1 with PIP2 vs. PBD-mutant form of ATP8b1 (FIG. 3K).

Unbiased RNAseq analysis of ATP8b1^{-/-} cells. To determine the effect of ATP8b1 on genome-wide transcriptional profile, the WT and ATP8b1^{-/-} HepG2 cells were subjected to an unbiased RNAseq analysis. The top altered pathways in ATP8b1^{-/-} hepatocytes included signal transduction, cell-adhesion, microvillus assembly, phospholipid translocation, response to LPS, and bile acid metabolism (FIG. 4A). As shown in FIG. 4B, the top altered genes included MASP1 (part of complement pathway, promotes inflammation) and ATP10A (regulated by cholesterol). These data indicate that cross talk between cell signaling pathways, bile acid metabolism, and phospholipid trafficking/metabolism in ATP8b1^{-/-} hepatocytes.

ATP8b1 is a negative regulator of Gasdermin D cleavage. Human patients carrying ATP8b1 mutations show extrahepatic manifestation such as pancreatitis, hearing loss, and atherosclerosis. Even after liver transplantation, which rescues hepatic symptoms, these extrahepatic symptoms persist. Inflammasome and Gasdermin D (GsdmD) pathways play a role in various inflammatory diseases, thus we tested role of ATP8b1 in regulating inflammasome activity. Strikingly, we found that stimulation with LPS alone was sufficient for cleavage of GsdmD in human ATP8b1^{-/-} macrophages as well as in monocytes (FIG. 5A). As expected, treatment of WT macrophages with LPS alone showed no GsdmD cleavage (FIG. 5A). Induction with secondary stimuli Nigericin led to the cleavage of GsdmD in both WT and ATP8b1^{-/-} macrophages, but the cleavage of GsdmD was much higher in ATP8b1^{-/-} cells (FIG. 5A). Nlrp3 inflammasome assembly in both macrophages and monocytes led to markedly higher IL-1β release from ATP8b1^{-/-} cells vs. WT cells (FIG. 5B, 5C). Interestingly, a significant increase in IL-1β release was observed in ATP8b1^{-/-} macrophages and monocytes upon treatment with LPS alone. As expected, the WT control showed no IL-1β release upon LPS stimulation (FIG. 5B, 5C).

To determine the mechanism of LPS-induced GsdmD cleavage, NF-kB localization and lysosomal membrane integrity were tested. The basal Nlrp3 expression was found to be higher in ATP8b1^{-/-} macrophages, but there was no difference in basal or LPS-induced nuclear localization of NF-kB in ATP8b1^{-/-} cells. Furthermore, there was no cytoplasmic release of lysosomal proteins such as Cathepsin B, a potent inducer of inflammasome activity (FIG. 5D). Also, no differences were found in basal cytoplasmic localization or starvation-induced nuclear localization of TFEB, a master regulator of lysosomal biogenesis. These data indicated that lysosomal generation and lysosomal membrane integrity were not compromised in ATP8b1^{-/-} cells. Though no differences were found in lysosomal membrane integrity, high resolution stimulated emission depletion (STED) microscopy showed a significantly lower lysotracker staining (˜80% reduction) in ATP8b1^{-/-} vs. WT macrophages (FIG. 5E). Lysotracker accumulates in acidic organelle, thus we tested if lysosomal pH was altered in ATP8b1^{-/-} macrophages. We used acridine orange, a metachromatic dye that emit green fluorescence in monomeric form and emit red fluorescence upon accumulation in form of stacks in acidic lysosomes. The lysosomal pH was significantly less acidic, with ˜55% reduction in red fluorescence per cell (n>40 cells per group) in ATP8b1^{-/-} vs. WT macrophages (FIG. 5F). To determine if ATP8b1 regulates GsdmD cleavage via canonical or non-canonical inflammasome pathway, we used MCC950, a potent inhibitor of Nlrp3 mediated canonical inflammasome pathway. As shown in FIG. 5G, the GsdmD was still cleaved in ATP8b1^{-/-} macrophages treated with LPS alone. Upon LPS+Nigericin treatment (canonical Nlrp3 pathway), MCC950 inhibited GsdmD cleavage by the Nlrp3 mediated component, but not the LPS only component (FIG. 5G). As expected, MCC950 completely abolished GsdmD cleavage in WT macrophages (FIG. 5G) These data indicate that though basal level of Nlrp3 are higher in ATP8b1^{-/-} cells, the cleavage of GsdmD is independent of Nlrp3.

To further elucidate the mechanism of induced GsdmD cleavage in ATP8b1^{-/-} cells, we tested if LPS can gain cytoplasmic access in ATP8b1^{-/-} cells. It's known that LPS entry into the cytoplasm, either via infection with gram-negative bacteria or via LPS electroporation, can lead to direct activation of caspase-11 (mouse) or 4/5 (humans) and subsequent GsdmD cleavage without engaging TLR4 signaling and downstream NF-kB activation. We found that Alexa488 labeled LPS penetrated plasma membrane and gained direct access to cytoplasm in ATP8b1^{-/-} monocytes, while no LPS was found in cytoplasm of WT cells under similar conditions. (FIG. 5H).

ATP8b1 regulate phagocytic and efferocytic activity of macrophages. Inflammation resolving pathways such as phagocytosis and efferocytosis play a major role in inflammatory diseases, and PIP2 is also involved in these processes. Since PFIC1 patients suffer from chronic inflammation, we determined phagocytic and efferocytic activity of ATP8b1-vs. WT macrophages. As shown in FIG. 6A, 6B, ATP8b1^{-/-} macrophages showed markedly reduced efferocytosis of apoptotic Jurkat cells vs. WT macrophages. The binding of apoptotic Jurkat cells to ATP8b1^{-/-} macrophages was not impaired (FIG. 6A), indicating downstream defects in efferocytic pathway. Interestingly, the expression of efferocytic receptor MerTK was higher in THP1-ATP8b1^{-/-} macrophages (FIG. 6C), indicating that reduced efferocytosis in ATP8b1^{-/-} macrophages is not due to reduced MerTK expression. The phagocytic activity of ATP8b1^{-/-} macrophages was assessed by efficacy to engulf FITC-labeled latex beads. As shown in FIG. 6D, ATP8b1^{-/-} macrophages showed markedly reduced phagocytosis vs. WT macrophages. The binding of latex beads to ATP8b1^{-/-} macrophages was not impaired (FIG. 6D), indicating that phagocytosis is impaired downstream of the binding step.

### Materials and Methods:

Cell Culture and Reagents. The RAW264.7, HepG2, THP-1, and HEK293 cells were from ATCC, and were cultured in appropriate media containing required growth factors and antibiotics.

Quantification of cell-surface PIP2. Cell surface PIP2 levels were determined by flow cytometry, confocal laser scanning microscopy, and fluorescent spectroscopy with SpectraMax® i3 Multi-Mode Detection Platform (Molecular Devices), using fluorescein conjugated Anti-PI(4,5)P2 IgM antibody (Z-G045 Echelon Biosciences).

PIP2 localization at the inner leaflet of the plasma membrane. HepG2-WT or HepG2-ATP8b1^{-/-} cells were stably transfected with fluorescent PIP2 reporter (2X-PH-PLC-eGFP, Addgene #35142) and PIP2 localization was determined by confocal microscopy and images were analyzed by using line-profile (ImageJ).

Fluorescence labeling of ATP8b1 protein. WT ATP8b1 or PBD mutant of ATP8b1 were labeled at the N-terminus using Zip-Alexa Fluor 488 labeling Kit (ThermoScientific, #Z11233) according to manufacturer's protocol.

Preparation of Large Unilamellar Vesicles (LUVs). Large unilamellar vesicles were prepared using extruder set with heating block (Avanti Polar lipid, USA, 610000 #) following manufacturer's protocol.

Surface Plasmon Resonance (SPR). Binding kinetics of PIP2 with different isoforms of ATP8b1 was analyzed using a BiacoreS200 instrument (Cytiva #29136649).

Microscale Thermophoresis (MST). MST experiments were performed to determine binding affinity (K_{d}) of ATP8b1 WT and ATP8b1 PBD mutant with PIP2. MST was measured using the Monolith NT.115 instrument (NanoTemper Technologies) and data was plotted using MO. Affinity analysis software version 2.2.7, (NanoTemper Technologies).

ATP8b1-PIP2 interaction using Giant Unilamellar Vesicles (GUVs). GUVs were formed by Poly-(vinyl) alcohol gel assisted method as described earlier, and lipid protein interaction was determined.

qRT-PCR assay. Quantitative real-time PCR amplification was performed using TaqMan Universal PCR Master Mix (Thermofisher, Catalog: 4304437) and gene-specific primers in a Quant Studio 3 Real-Time PCR system (Applied Biosystems).

Western Blotting. Western blot analysis was performed on protein extracts prepared using MPER lysis buffer±various treatment conditions.

Indirect Immunofluorescence. THP-1 cells were grown in iBidi chamber slides and differentiated into macrophages and probed with Cathepsin B (cell signaling #31718), MerTK (ThermoFisher Scientific; PA5-15028), TFEB (Proteintech #13372-I-AP) or NF-kB (cell signaling #4764) antibodies.

Efferocytosis Assay. THP-1 monocytes were differentiated into macrophages in indicated media for 70-80% confluency. Jurkat cells (ATCC) were labeled with 1 μM calcein and treated with 1 μM of staurosporine to induce apoptosis, followed by efferocytosis assay.

IL-1β ELISA. THP-1 cells were treated with LPS (1 μg/ml) for 3 hours and with Nigericin (5 μM) for 1 hour. After treatment, media was collected and spun at 12000 RPM for 5 minutes. Then, the supernatant was collected and IL-1β was measured using a human-specific IL-1β ELISA kit (R&D system #DLB50).

Acridine Orange Staining. THP-1 macrophages were treated with acridine orange stain solution (Sigma #A9231) for 10 minutes at 37° C. Cells were imaged using confocal microscopy (excited with blue light (488 nm) with emission bandpass at wavelengths green (480-560) nm and red (590-660) nm, respectively.

Lysotracker staining. Lysotracker staining was performed using lysotracker deep red dye (Invitrogen #L12492) and following manufacturer's instructions. THP-1 cells were incubated with 75 nM lysotracker deep red dye (Invitrogen #L12492) at 37° C. for 2 hours, followed by fixation using methanol-free 3.7% paraformaldehyde and images were acquired using STEDYCON microscopy (Nikon Eclipse T2 assembled with Abberior instrument, USA), with excitation 405 nm for DAPI, and 561 nm for lysotracker.

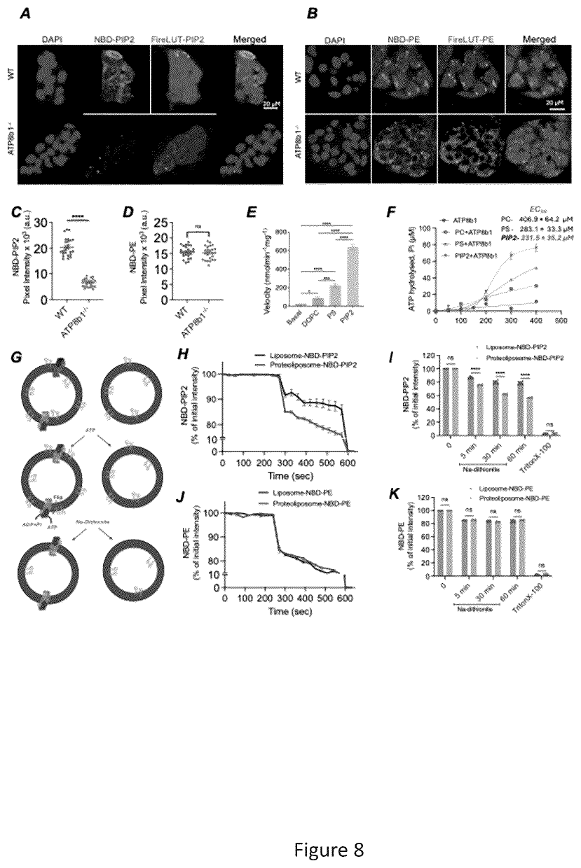

To further test the role of flippase in PIP2 translocation inside the cells, rather than endocytosis, a recently reported confocal-microscopy-based phospholipid flip assay was used. As shown in FIG. 8A, WT-HePG2 cells showed an intense fluorescent signal inside the cell after BSA wash, indicating robust PIP2 flip. In contrast, ATP8B1^{-/-} cells showed a weak signal at the PM in the form of puncta after BSA wash (FIG. 8A), reminiscent of endocytosis rather than ATPase-mediated active PIP2 flip. The flip of PE was unaffected in ATP8B1^{-/-} cells (FIG. 8B). The quantification of flipped NBD-PIP2 showed ˜4-fold reduction in HepG2-ATP8B1^{-/-} vs. WT cells (FIG. 8C), while no differences were found in the flip of NBD-PE (FIG. 8D).

ATP8B1 deletion may affect PIP2 translocation due to changes in membrane properties or by indirectly influencing the activity of other proteins involved in lipid trafficking. To determine if ATP8B1 can directly flip PIP2, a cell-free system was reconstituted. The GST-tagged human ATP8B1 protein was cloned in a baculovirus, followed by transduction of insect Sf9 cells, and expression was confirmed by western blot using anti-GST antibody. The ATPase activity of full-length ATP8B1 was determined in the presence of various phospholipids, and a notable increase in ATPase activity was observed with 300 μM PIP2 (FIG. 8E). The EC₅₀ values of PIP2 and PS-stimulated activity of ATP8B1 were 231.5±35.2 μM and 283±33.3 μM, respectively (FIG. 8F).

The EC₅₀ values of PC-stimulated activity of ATP8B1 was 1.75-fold higher than PIP2 (FIG. 8F). Next, we utilized reconstituted ATP8B1-proteoliposomes with NBD-labeled phospholipids and sodium dithionite quenching assay to measure phospholipid flip (FIG. 8G), as described before. A marked decrease in the fluorescence intensity of NBD-PIP2 was observed following dithionite quenching of ATP8B1 containing proteoliposomes vs. control liposomes, with ˜12% NBD-PIP2 flip in 5 min, ˜22% NBD-PIP2 flip in 30 min, and ˜27% NBD-PIP2 flipped in 60 min (FIG. 8H, 8I). No significant changes were observed in the flip of NBD-PE, indicating that ATP8B1 does not play a direct role in the flip of PE (FIG. 8J, 8K).

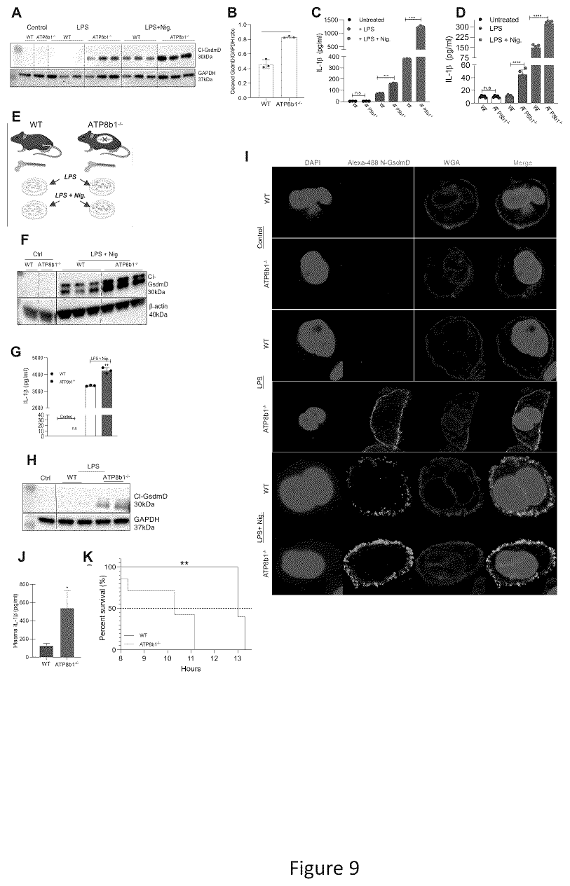

ATP8B1 regulates GSDMD cleavage and the formation of membrane pores: GSDMD promotes various inflammatory diseases by regulating the release of pro-inflammatory cytokines. Strikingly, we found that LPS exposure alone was sufficient to trigger GSDMD cleavage in human ATP8B1^{-/-} monocytes and macrophages vs. WT controls (FIG. 9A). Induction with secondary stimuli Nigericin led to the cleavage of GSDMD in both WT and ATP8B1^{-/-} monocytes, but the cleavage of GSDMD was much higher in ATP8B1^{-/-} monocytes (FIG. 9A,9B). LPS alone or LPS+Nigericin treatment led to markedly higher ILIβ release from ATP8B1^{-/-} monocytes and macrophages vs. WT controls (FIG. 9C, 9D). Similar trends were observed in primary bone marrow-derived macrophages (BMDMs), where LPS+Nigericin treatment led to significantly higher GSDMD cleavage and IL1b release in BMDMs isolated from Atp8b1^{-/-} mice vs. WT mice (FIG. 9E, 9F). Importantly, similar to human ATP8B1^{-/-} immune cells, exposure to LPS alone induced cleavage of GSDMD in BMDMs isolated from Atp8b1^{-/-} mice vs. WT mice (FIG. 9H). To determine if cleavage of GSDMD indeed leads to physiological consequences, the status of GSDMD membrane pores in ATP8B1^{-/-} vs. WT cells was assessed. As expected, the untreated WT or ATP8B1^{-/-} cells showed no GSDMD pores on the PM. Exposure to LPS alone led to robust GSDMD pores on the PM of ATP8B1^{-/-} cells, while no GSDMD pores were observed in WT cells (FIG. 7I). Furthermore, the ATP8B1^{-/-} cells showed markedly increased GSDMD membrane pores vs. WT cells upon assembly of NLRP3 inflammasome by LPS+Nig. treatment (FIG. 9I).

To determine if the Atp8b1^{-/-} mice are more susceptible to LPS-induced inflammation, a lethal dose of LPS was injected in WT and Atp8b1^{-/-} mice. As shown in FIG. 9J, the plasma IL1b levels were ˜4-fold higher in Atp8b1^{-/-} mice injected with LPS vs. WT mice. Furthermore, the Atp8b1^{-/-} mice showed a mean survival rate of only ˜10.30 h vs. ˜13 h of WT mice (FIG. 9K), indicating increased susceptibility of PFICl mice to sepsis-induced mortality.

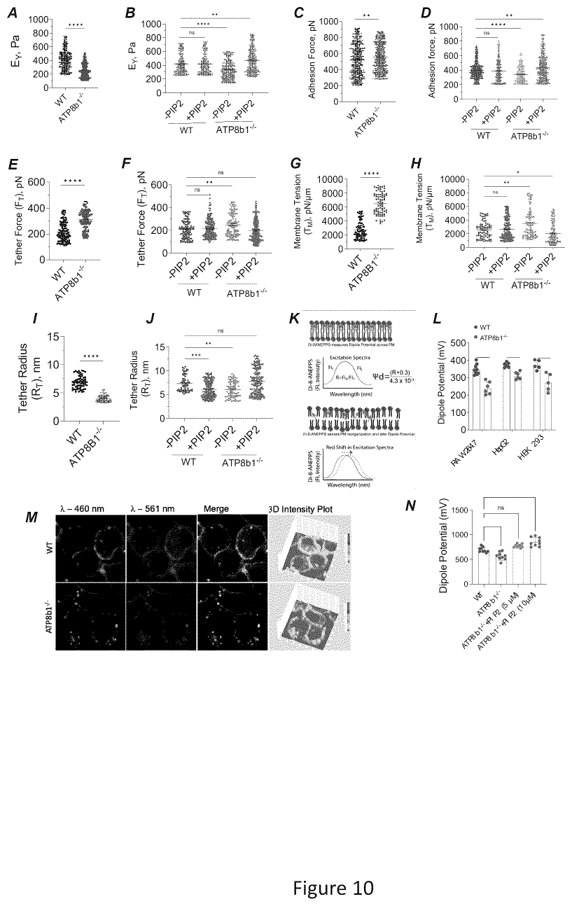

Altered biomechanical properties of ATP8B1^{-/-} PM can be restored by PIP2: Phagocytosis and efferocytosis are shown to be affected by the biophysical properties of cell membrane. Atomic Force Microscopy (AFM) analysis revealed that the biomechanical properties of WT THP-1 macrophages were: Young's modulus (E_{Y})=415.1±141.7 Pa, adhesion force (F_{ad})=518.8±196.1 pN, tether force (Fr)=213.3±72.8 pN, apparent membrane tension (T_{M})=2705 ±1254 pN/μm, and radius of tether (R_{T})=6.9±2 nm, which were used as reference compared to test cases. Significant decreases in E_{Y} and R_{T}, and concomitant increases in F_{ad}, F_{T}, and T_{M}, were noted in ATP8B1^{-/-} cells compared to WT cells (FIG. 10A-10I). To investigate if altered biomechanical properties are due to the altered distribution of PIP2 across plasma membrane bilayer in ATP8B1^{-/-} cells, PIP2 was supplemented via liposomes. Unlike non-micellar PIP2, PIP2 liposomes can diffuse in the PM and distribute equally to both sides of the PM. PIP2 delivery from DOPC (denoted as +PIP2) did not significantly alter the E_{Y}, F_{ad}, F_{T}, and T_{M} of WT cells compared to the delivery of DOPC alone (denoted as −PIP2) or compared to the WT cells that did not receive DOPC (FIG. 10B-10J). Interestingly, the average E_{Y} and R_{T} significantly increased, while F_{T}, T_{M} and F_{ad} values significantly decreased, in ATP8B1^{-/-} macrophages with the addition of DOPC±PIP2 (p<0.001 vs. ATP8B1^{-/-} in all the cases; p<0.001 for ATP8B1^{-/-}+PIP2 vs. ATP8B1^{-/-}−PIP2) (FIG. 10B-10J). We evaluated if osmotic pressure differences across the membrane (ΔP) might be contributing to the changes in tether forces. The membrane tension on the cell could be related to □P using the Young-LaPlace equation (ΔP=(2T_{M}/(R_{T}). For the 2 nN force imparted on the cells by the AFM beaded-tip, the ΔP values for WT and ATP8B1^{-/-} cells were 772.8±37 kPa and 2999±79 kPa, respectively (p<0.001 for WT vs. ATP8B1^{-/-}), suggesting a significant increase in osmotic pressure difference across the plasma membrane in ATP8B1 knockout cells. Addition of DOPC±PIP2 did not affect ΔP in WT cells (p>0.1 vs. WT). However, the □P values were 1071.4±80.9 kPa and 464.7±27 kPa in ATP8B1^{-/-} cells with the addition of DOPC-PIP2 and DOPC+PIP2 liposomes, respectively, suggesting that the addition of PIP2 dropped the ΔP levels similar to that in WT cells. A similar trend was observed in HEK293 cells, where deletion of ATP8B1 decreased Young's modulus (E_{Y}), indicating more elastic PM vs. WT cells. These data were further confirmed in various ATP8B1^{-/-} cell lines by measuring the dipole potential of PM by using Di-8-ANEPPS dye (FIG. 10K). The dipole potential of PM in ATP8B1^{-/-} cells was significantly lower vs. corresponding WT cells (FIG. 10L). We also visualized the distribution of Di-8-ANEPPS dye via Stimulated Emission Depletion Super-Resolution Imaging (STEDYCON) microscopy using dual-wavelength ratiometric imaging approach. The 3D surface plot depicting the fluorescence intensity of Di-8-ANEPPS illustrated a more localized presence of the dye on the PM of WT cells vs. diffused distribution in ATP8B1^{-/-} cells (FIG. 10M). To determine if the change in dipole potential is due to PIP2 redistribution in ATP8B1^{-/-} cells, cells were supplemented with PIP2 liposomes. As shown in FIG. 10N, PIP2 supplementation restored membrane dipole potential in a dose-dependent manner.

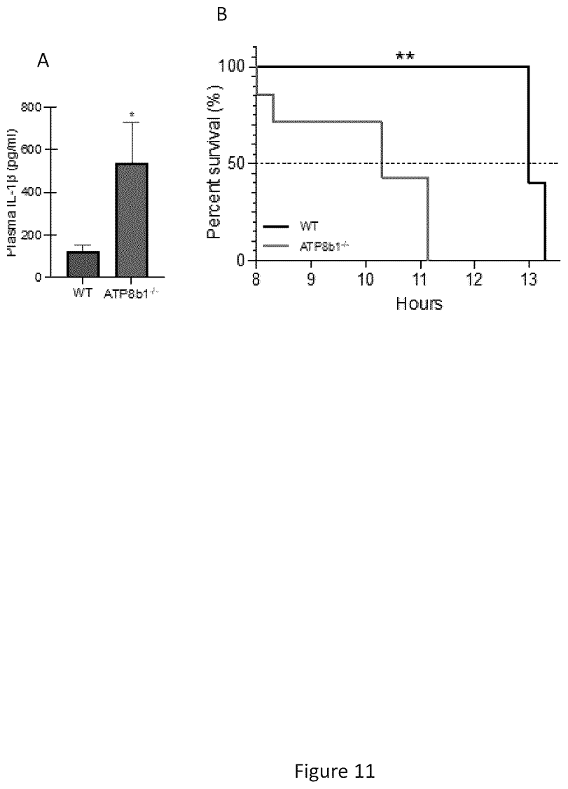

To determine if the Atp8b1 knockout mice are more susceptible to LPS-induced inflammation, a lethal dose of LPS was injected in WT and Atp8b1−/− mice. As shown in FIG. 11A, the plasma IL1b levels were ˜4-fold higher in Atp8b1−/− mice injected with LPS vs. WT mice. Furthermore, the Atp8b1−/− mice showed a mean survival rate of only ˜10.30 h vs. ˜13 h of WT mice (FIG. 11B), indicating increased susceptibility of Atp8b1^{-/-} KO PFIC1 mice to sepsis-induced mortality.

The ATP8b1 KO mouse strain used for this research project, B6.129S4-Atp8b1tm1Nbf/Mmucd, RRID:MMRRC_036310-UCD, was obtained from the Mutant Mouse Resource and Research Center (MMRRC) at University of California at Davis, an NIH-funded strain repository, and was donated to the MMRRC by Laura Bull, Ph.D., University of California, San Francisco.

What has been described above includes examples of one or more embodiments. It is, of course, not possible to describe every conceivable modification and alteration of the above compositions or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

1. A method of treating, preventing, inhibiting, or ameliorating the symptoms of a disease or disorder that is modulated or otherwise affected by ATPB81 levels, the method comprising administering to an individual in need thereof a therapeutically effective amount of at least one of a GsdmD blocker, a PIP2 inhibitor, and an antisense oligonucleotide to an individual in need thereof.

2. The method of claim 1, wherein the disease or disorder is selected from inflammation, extra-hepatic inflammatory clinical features, such as steatohepatitis, fat malabsorption, efferocytosis, pancreatitis, sporadic hearing loss, Alzheimer's disease, diarrhea, pancreatitis, and atherosclerosis.

3. The method of claim 1, wherein the disease or disorder is efferocytosis.

4. The method of claim 1, wherein the disease or disorder is hepatic inflammation.

5. The method of claim 1, wherein the individual in need thereof is selected from a child, teen, adult, and an elderly adult.

6. The method of claim 1, wherein the GsdmD blocker is selected from the group consisting of disulfiram and dimethyl fumarate.

7. The method of claim 6, wherein the GsdmD blocker is disulfiram.

8. The method of claim 1, wherein the PIP2 inhibitor is selected from the group consisting of ISA2011b, IC-87114, BKM120, CAL-101, and LY 294002.

9. The method of claim 8, wherein the PIP2 inhibitor is ISA2011b.

10. The method of claim 1, wherein the antisense oligonucleotide is an antisense oligonucleotide targeting PIP2 biosynthetic enzyme PIP5K1a.

11. The method of claim 1, wherein administering a therapeutically effective amount results in a reduction in at least one of fat malabsorption, fatty diarrhea, and cholesterol present in feces of the individual.

12. A method of treating, preventing, inhibiting, or ameliorating the symptoms of progressive familial intrahepatic cholestasis 1 (PFIC1) in an individual in need thereof, the method comprising administering a therapeutically effective amount of at least one of a GsdmD blocker, a PIP2 inhibitor, and an antisense oligonucleotide to an individual in need thereof.

13. The method of claim 12, wherein the GsdmD blocker is selected from the group consisting of disulfiram and dimethyl fumarate.

14. The method of claim 13, wherein the GsdmD blocker is disulfiram.

15. The method of claim 12, wherein the PIP2 inhibitor is selected from the group consisting of ISA2011b, IC-87114, BKM120, CAL-101, and LY 294002.

16. The method of claim 15, wherein the PIP2 inhibitor is ISA2011b.

17. The method of claim 12, wherein the antisense oligonucleotide is an antisense oligonucleotide targeting PIP2 biosynthetic enzyme PIP5K1a.

18. A method of reducing inflammation in an individual suffering from symptoms of progressive familial intrahepatic cholestasis 1 (PFIC1), the method comprising administering a therapeutically effective amount of at least one of a GsdmD blocker, a PIP2 inhibitor, and an antisense oligonucleotide to the individual in need thereof.

19. The method of claim 1, wherein the GsdmD blocker is disulfiram, the PIP2 inhibitor is ISA2011b, and the antisense oligonucleotide targets the PIP2 biosynthetic enzyme PIP5K1a.
